(19) United States
(12) Patent Application Publication
(10) Pub. No.: US 2019/0021886 A1
(43) Pub. Date: Jan. 24, 2019

(54) SEGMENTED SELF-EXPANDING STENT

(71) Applicant: Cook Medical Technologies LLC, Bloomington, IN (US)

(72) Inventors: Seoggwan Kim, West Lafayette, IN (US); Emma Moran, West Lafayette, IN (US); Richard A. Swift, South Bend, IN (US); Keith Milner, West Lafayette, IN (US)

(21) Appl. No.: 16/033,561

(22) Filed: Jul. 12, 2018

Related U.S. Application Data
(60) Provisional application No. 62/534,360, filed on Jul. 19, 2017.

(51) Int. Cl. A61F 2/82; A61F 2/958

(52) U.S. Cl. CPC A61F 2/82; A61F 2002/9511; A61F 2002/826; A61F 2/958

(57) ABSTRACT

Disclosed herein is a delivery system and method for delivering a plurality of separate intraluminal support devices with predictable spacing. The intraluminal support devices may be self-expanding devices mounted over an inflatable balloon of a balloon catheter and adhered thereto by an adhesive. The intraluminal support devices may optionally have a flattened tip region.

### CROSS REFERENCE TO RELATED APPLICATIONS

This application claims the benefit of U.S. Provisional Patent Application No. 62/534,360, filed on Jul. 19, 2017, the entire contents of which is hereby incorporated by reference.

### BACKGROUND

The present application generally relates to medical devices. More particularly, the present application relates to device assemblies and delivery arrangements for spacing a plurality of implantable, segmented support devices to a body vessel in need of treatment.

Stents and other implantable medical devices which incorporate stents are in widespread use in the medical field for dilating patients' vessels, for closing off aneurysms, for treating vascular dissections, for supporting prosthetic elements and so on. Stents have the function of holding the vessel open or for holding a device securely against the vessel wall to effect a good seal as well as to prevent device migration. As a result, it is desirable for the stent to be able to apply an opening force and to do so reliably. Self-expanding implants, in particular, are selected for such applications due to the assurance that they will expand to fit the vessels in which they are implanted, allow for less-complicated delivery schemes, and have a tendency to remain in the specific location to which they have been delivered. However, in many instances an implant such as a stent is constructed as a relatively long device with longitudinally-linked radial structures.

An improvement to an intravascular support device, such as a stent, could involve a design made up of a series of independent flexible rings which are not interconnected and that provide radial support without impeding or altering the physiologic axial compression and bending of the vessel in which they are implanted, particularly during ambulation and positional changes. However, designs with increased radial force are generally accompanied by a reduction in flexibility. For instance, in a particular design, a 200% increase in radial force (from 0.4 Newton per millimeter (N/mm) to 1.55 N/mm) yielded a bending stiffness from 0.0027 N/mm to 0.0119 N/mm, and an axial stiffness from 0.0373 N/mm to 0.1890 N/mm, an increase of 400% in each of these dimensions.

Although axially connected devices have a dimension of added inflexibility, their unitary nature allows for improved placement and alignment at delivery, as the positioning of one radial portion of the device is dictated by the position of the adjacent radial portion and the length of the connector between the two. Devices delivered as separate segments do not possess connectors that dictate such spacing.

It has been a challenge to develop delivery assemblies and schemes for delivering a plurality of separate intraluminal support devices with reliable, predictable spacing therebetween.

### SUMMARY

In one aspect, the present disclosure provides a medical device medical device assembly including a balloon catheter. The balloon catheter includes a catheter body extending from a proximal end to a distal end and defining a longitudinal axis therethrough, and a balloon inflatable from a deflated state to an inflated state disposed circumferentially around the catheter body. The assembly includes a plurality of separate intraluminal support devices for implantation into a lumen of a body vessel. Each intraluminal support device may have a tubular body comprising at least one ring which is radially expandable from a compressed state to an expanded state. Each intraluminal support device is a self-expanding device. The plurality of intraluminal support devices is releasably secured about a delivery portion of the balloon when the balloon is in the deflated state. The plurality of intraluminal support devices is arranged such that a portion of one of the intraluminal support devices is in contact with a portion of an adjacent intraluminal support device. The plurality of intraluminal support devices are detached from the balloon when the balloon is inflated to an inflated state.

In another aspect, the present disclosure provides a medical device assembly. The assembly includes a balloon catheter which has a catheter body extending from a proximal end to a distal end and defining a longitudinal axis therethrough. The balloon catheter also includes a balloon inflatable from a deflated state to an inflated state disposed circumferentially around the catheter body. The assembly incorporates a plurality of separate self-expanding intraluminal support devices for implantation into a lumen of a body vessel. Each intraluminal support device includes a tubular body of at least one ring and is radially expandable from a compressed state to an expanded state. Each ring includes a plurality of struts and bends arranged in a repeating pattern to define a plurality of peaks. Each of the plurality of peaks defines a flat surface in a plane perpendicular to the longitudinal axis. The assembly may include a covering attached to and disposed over each of the plurality of intraluminal support devices. The plurality of intraluminal support devices are releasably secured about a delivery portion of the balloon in the deflated state such that at least one of the plurality of peaks of one of the intraluminal support devices is in contact with at least one of the plurality of peaks of an adjacent intraluminal support device. The plurality of intraluminal support devices become detached from the balloon when it is in the inflated state. The delivery portion of the balloon is substantially cylindrical in the inflated state.

In another aspect, the present disclosure provides method of making a medical device assembly. The method includes a step of aligning a plurality of self-expanding intraluminal support devices each being made up of at least one ring, the ring including a plurality of struts and bends arranged in a repeating pattern to define a plurality of peaks, each device defining a lumen therethrough, over a mandrel. The mandrel has a substantially cylindrical body having an outer surface. A plurality of pins extend outwardly from the outer surface, such that the plurality of pins constrain the plurality of intraluminal support devices in a predetermined arrangement involving peak-to-peak contact between the intraluminal support devices. In another step, the mandrel is removed such that the plurality of intraluminal support devices remain in the predetermined arrangement. In a further step, the method includes inserting a balloon catheter through each lumen. The balloon catheter includes a catheter body extending from a proximal end to a distal end and defining a longitudinal axis therethrough, and a balloon inflatable from a deflated state to an inflated state disposed circumferentially around the catheter body. The balloon has a delivery portion for retention and delivery of the plurality of intraluminal support devices. In another step, the method includes compressing the plurality of intraluminal support devices over the balloon in the deflated state.

Further objects, features and advantages of this system will become readily apparent to persons skilled in the art after a review of the following description, with reference to the drawings and claims that are appended to and form a part of this specification.

### BRIEF DESCRIPTION OF THE DRAWINGS

FIG. 1 is a side view of a first embodiment of an implantable intraluminal support device segment constructed in accordance with the principles of the present invention;

FIG. 2 is a side view of another embodiment of an implantable intraluminal support device segment constructed in accordance with the principles of the present invention;

FIG. 3A is a side view of another embodiment of an implantable intraluminal support device segment constructed in accordance with the principles of the present invention;

FIG. 3B is a close-up view of a device end of the device illustrated in FIG. 3A;

FIG. 4A is a side view of a plurality of intraluminal support device segments in a compressed arrangement in accordance with one embodiment of the present invention;

FIG. 4B is a side view of a plurality of intraluminal support device segments in an expanded arrangement;

FIG. 4C is a side view of a plurality of intraluminal support device segments in a compressed arrangement in accordance with another embodiment of the present invention;

FIG. 4D is a side view of a plurality of intraluminal support device segments arranged about an inflatable balloon in accordance with another embodiment of the present invention;

FIG. 5A is a side view of a plurality of intraluminal support device segments in a compressed arrangement in accordance with another embodiment of the present invention;

FIG. 5B is a side view of a plurality of intraluminal support device segments in an expanded arrangement;

FIG. 5C is a side view of a plurality of intraluminal support device segments in a compressed arrangement in accordance with another embodiment of the present invention;

FIG. 6 is a perspective view of a mandrel for the alignment of intraluminal implants according to the principles of the present disclosure;

FIG. 7 is a perspective view of the mandrel of FIG. 6 with a segmented device being aligned thereon;

FIGS. 8A and 8B are embodiments of a segmented device according to the present disclosure having a number of different partial coverings formed thereon; and

FIG. 9 is an embodiment of a segmented intraluminal device formed with a complete covering applied thereto.

### DETAILED DESCRIPTION

The drawings are purely schematic illustrations of various aspects of the invention and are not necessarily to scale, unless expressly stated.

The terms “substantially” or “about” used herein with reference to a quantity includes variations in the recited quantity that are equivalent to the quantity recited, such as an amount that is equivalent to the quantity recited for an intended purpose or function. “Substantially” or derivatives thereof will be understood to mean significantly or in large part.

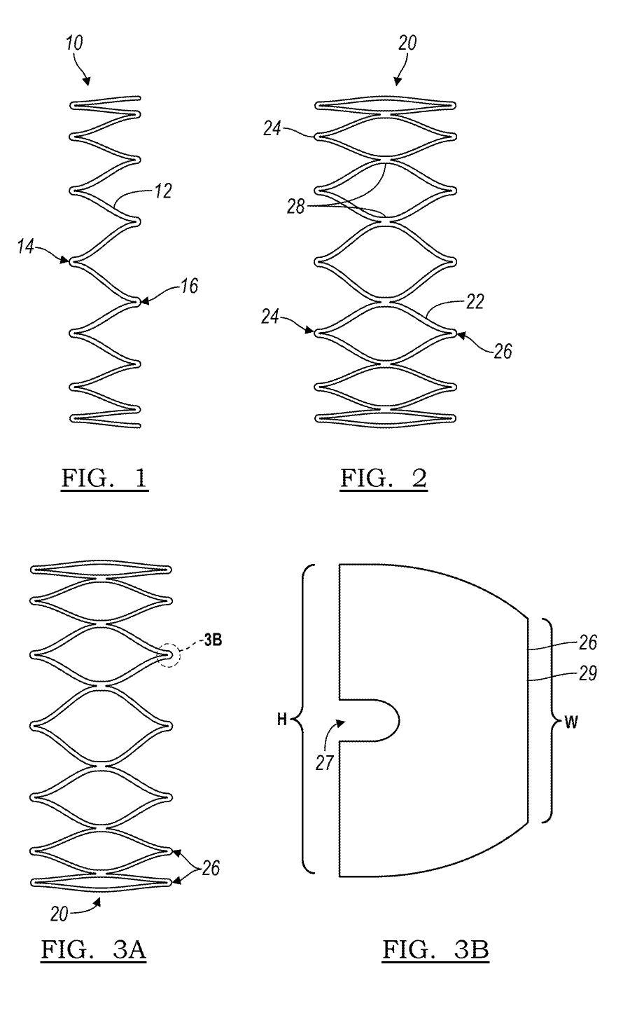

FIG. 1 illustrates an intraluminal support device 10 formed as a single ring comprising a zigzag pattern of struts 12 that meet at a series of first peaks 14 and second peaks 16 on the opposite side of the device 10, longitudinally, of first peaks 14. Devices made of a series of rings with a structure similar to that depicted in FIG. 1, interconnected by longitudinally elements that stretch from a peak of one ring to a peak of an adjacent ring in order to axially align the plurality of rings, are commercially available.

Similarly, FIG. 2 depicts a device 20 which resembles two of device 10 of FIG. 1 placed in-phase but in an opposing orientation in order to yield a single device 20. The V-shaped structures are made of struts 22 which form a series of bends that give rise to first peaks 24 and second peaks 26. Each strut meets at a junction 28 in the longitudinal center of the device 20. The junctions 28 are substantially aligned with one another as the circumference of the device 20 is traced.

The device according to the principles of the present invention is a self-expanding device. Such devices are expandable from a collapsed or compressed configuration to an expanded configuration. Such a device may be made from a biocompatible material, or a material which is able to be made biocompatible. Examples of suitable materials include, without limitation, nickel—titanium alloys, cobalt—chromium alloys, nickel—chromium alloys, nickel—cobalt—chromium alloys, nickel—cobalt—chromium—molybdenum alloys, nickel—titanium—chromium alloys, and other shape memory and/or superelastic materials, including alloys which include at least one of molybdenum, tantalum, titanium, palladium, and platinum. Polymers and composite materials may also provide the properties necessary for making such a device.

Loading of pluralities of segmented intraluminal support devices such as device 10 and device 20 as depicted in FIGS. 1 and 2 can be challenging, because each segment is not connected with a neighboring segment. This is particularly evident when the devices are loaded into a compression apparatus, such as a stent crimper. When using a conventional loading technique, that is, compressing the device radially to the delivery system diameter using a crimper, and then pushing the device out of the crimper to be transferred to a sheath, pushing force does not transfer well (if at all) owing to the lack of interconnectivity between rings.

In order to combat this issue of lack of pushing force transfer, FIG. 3A illustrates a variation of the device 20 as shown in FIG. 2. In this embodiment, the first peaks 24 and second peaks 26 have been constructed to have a flat profile. As can best be seen in the close-up view of FIG. 3B, the peak 26 has a flat end 29 opposite the interior of the bend 27. The width W of the flattened tip end 29 is greater than a width of a typical peak or bend of a zigzag style device ring. The flattened tip ends 29 allow for greater contact between adjacent devices, thereby increasing the ease with which they might be expelled from the compression device. In some embodiments, the width W may be as wide as the height H of the tip, allowing for an even greater area of contact between separate device segments. The width W may be about 1.2 to about 2.5 times as wide as the width of a strut, or any value in between, such as about 1.5 times as wide as a strut width. The tip may include a rounded portion which transitions into the flattened surface.

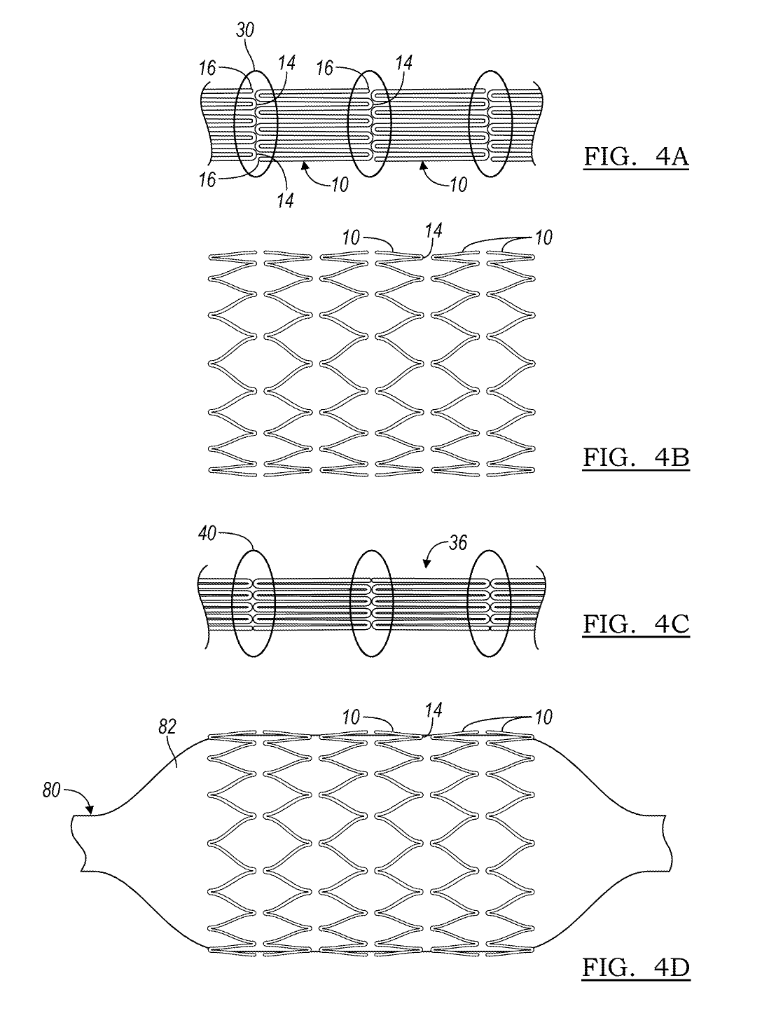

Another technique to overcome the difficulties in removing compressed devices from a crimper may, in some embodiments of the invention, involve compressing and adhering the plurality of intraluminal support devices over the inflatable balloon of a balloon catheter. Typically, self-expanding devices are not delivered to the area to be treated via a balloon catheter, owing to the fact that there is no need for an inflatable element; when the self-expanding devices are no longer constrained, such as by an outer sleeve or sheath of the delivery apparatus, the shape memory properties of the device allow it to expand on its own without the assistance of an external inflation apparatus. However, due to the aforementioned challenge associated with removing a plurality of crimped devices from a compression device, and in order to maintain relative spacing of the plurality of devices during delivery to the lumen of a body vessel, such a delivery scheme may be employed. A plurality of self-expanding devices 10 over a partially-inflated balloon 82 of a balloon catheter 80 are depicted in FIG. 4D.

The balloon catheter 80 can be of a variety of designs. In some embodiments, balloon catheters that can be used in an assembly of the present invention include those that have a higher coefficient of friction outer surface on the balloon portion, or a surface which is more adhesive or relatively sticky, such that it is more straightforward to hold the device segments in place. The portion of the balloon over which the ring-shaped support devices are mounted is referred to as the delivery portion. In one embodiment, the delivery portion, when inflated, takes on a substantially cylindrical shape.

Because self-expanding devices are biased to expand when unconstrained, an adhesive may be employed to maintain the devices in compressed, or partially compressed, configuration after crimping and during delivery. An adhesive for use in this application is biocompatible, having no byproduct that causes harm or injury to the body, and has sufficient strength to restrain the device on the balloon while the balloon is in the deflated state, but which is frangible upon expansion or inflation such that the devices easily release from the balloon to which they were formerly adhered. Materials that can be used for such adhesion include, but are not limited to, biodegradable adhesives, including those sold by RS Industrial under the trade name ECO-SQUARES.

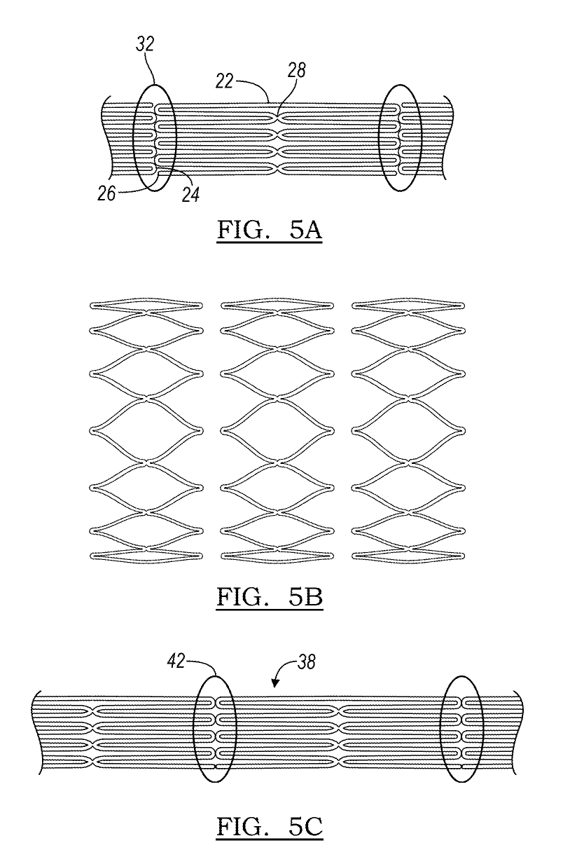

While a device 10/20 having at least some peaks or bends including a flattened tip end have been previously described herein, other alignment strategies may assist in allowing for a plurality of self-expanding devices to exit a compression device and/or maintain their alignment during delivery and implantation. Such arrangements may be seen in FIGS. 4A-4C and 5A-5C, and are detailed below.

In FIG. 4A, single-ring devices 10 of a construction as illustrated in FIG. 1 are illustrated in a collapsed configuration, as they would be placed over a deflated balloon (not shown.) The plurality of devices 10 is arranged with the first peaks 14 of one device being in contact with the second peaks 16 of an adjacent device to form contact interface 30. Contact interface 30 is formed as an out-of-phase or offset alignment of the peaks of one device as compared to the next device. That is, if one device 10 is considered to be in a first position, the second device adjacent the first device will be rotated about the longitudinal axis of the device assembly such that the extreme end of the peak is not in contact with the extreme end of the peak of the neighboring device. The offset and resulting contact may be improved by adopting a flattened-end device as is described by FIG. 3B, so that the area of contact between rings is greater, so that each peak will distribute its force to two other peaks of an adjacent ring, and so that more pushing force between rings will be realized when exiting a compression apparatus.

FIG. 5A illustrates an offset arrangement as illustrated in FIG. 4A, except with the ring structure bearing diamond-shaped strut arrangements of FIG. 2. In this embodiment, the first peaks 24 are offset from the second peaks 26 of an adjacent device at interface 32. The spacing of the devices 10/20 when the balloon over which they are mounted is inflated and expanded can be seen in FIGS. 4B and 5B, respectively.

In another embodiment, the plurality of devices can be axially aligned in phase rather than out of phase. FIG. 4C shows such an arrangement of devices 10, with the first peaks 14 of one device aligning substantially entirely similarly with the second peaks 16 of an adjacent device. Similarly, FIG. 5C illustrates an equivalent arrangement in which the units are devices 20 rather than devices 10. The direct peak-to-peak contact can be useful for a device or stent of standard construction, but may have added benefit when used in conjunction with a device having a flattened end as in FIG. 3B.

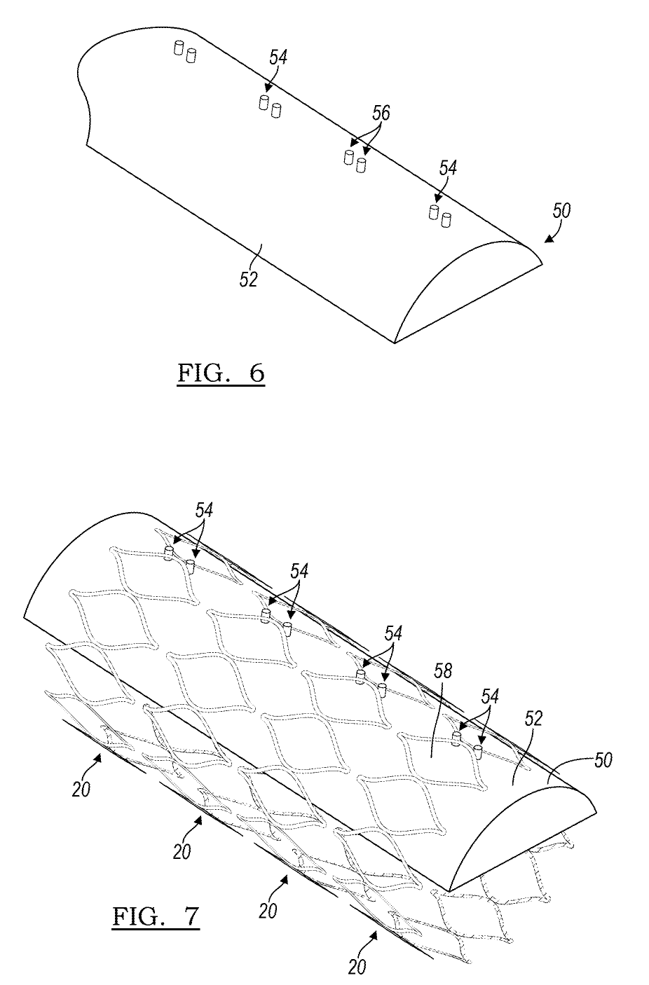

FIG. 6 shows a mandrel 50 which may be used for aligning ring-shaped support devices according to the principles of the present disclosure. The mandrel has a truncated, partial-cylindrical body 52 and is studded with a plurality of pins 54 arranged in a row parallel to the longitudinal axis of the cylinder on which the shape of the mandrel 50 is based. In some embodiments, the pins 54 may be arranged in pairs 56 in order to retain peak structures of the devices attached thereto. The mandrel 50 is employed to configure the plurality of devices into an initial alignment, after which they are placed into the crimper or other compression apparatus, and the mandrel 50 removed. FIG. 7 illustrates a plurality of devices 20 placed about the outer surface 58 of mandrel 50 and secured by pins 54. Such a mandrel 50 may also be used for aligning devices 10 of FIG. 1. The truncated cylinder shape of mandrel 50 allows for its straightforward removal from the lumens of the separate devices after use.

Instead of pins 54 extending from outer surface 58 of a mandrel 50, other means of attachment of the devices to the mandrel 50 may instead be employed. For instance, the devices 10/20 may be attached to the mandrel 50 via sutures, clips, or any other suitable attachment. Additionally, the mandrel 50 may be of a different design than illustrated in FIGS. 6 and 7, so long as it can be easily withdrawn from the devices after securing the devices at the predetermined spacing.

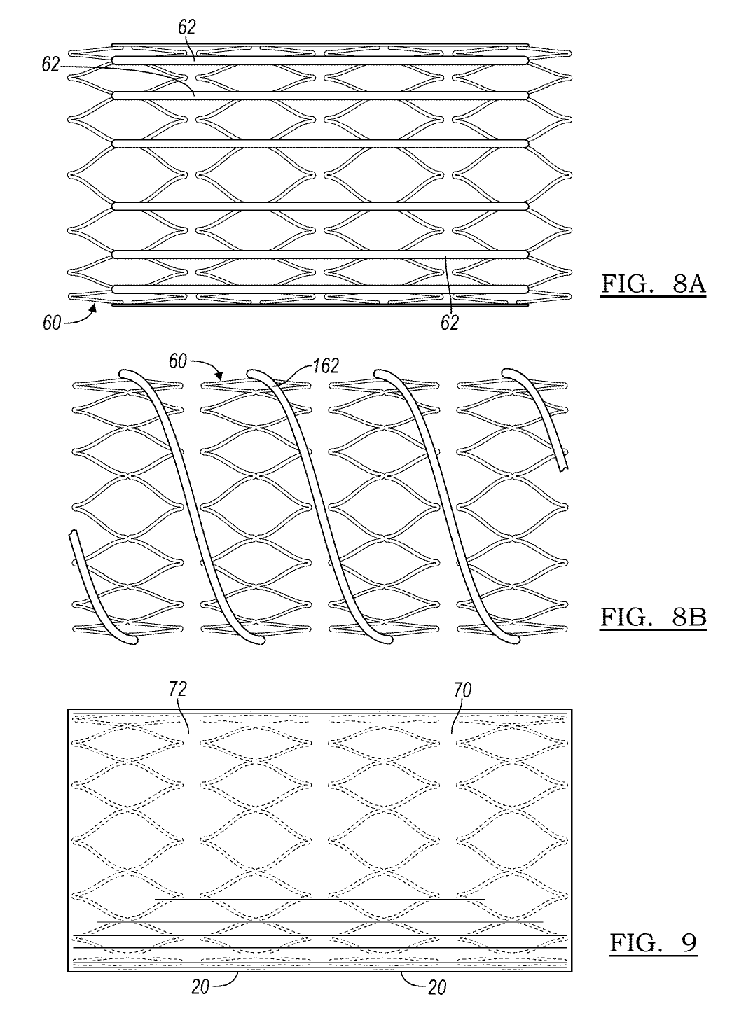

In some applications, it may be desirable to assist in maintaining the axial spacing of a plurality of devices by using a covering to retain the devices in relative position. FIGS. 8A and 8B show examples of a partial covering 62/162 which may be employed with a plurality of devices 60. In FIG. 8A, the coverings 62 are strips that run parallel to the longitudinal axis of the device and extend over the outer surface thereof, connecting to peaks of the rings of the devices 60. In FIG. 8B, the coverings 162 are instead spiral or helical coverings that extend about the outside of the devices. Other shapes and configurations of partial coverings are within the scope of the present disclosure. Use of a covering, in some embodiments, may obviate the need for a mandrel when crimping the device, as the covering maintains the spacing between rings.

FIG. 9 illustrates a complete covering 70 that represents a substantially cylindrical sleeve that attaches to and extends over the plurality of rings 20. The covering 70 then forms an outer surface 72 for the plurality of devices. Although the structure of the composite device 20/70 appears to approximate that of a stent graft as is known in the art, the material of the cover 70 allows for disposition of the rings 20 over an inflatable balloon of a balloon catheter in such a way that they are in contact when the balloon is in the deflated state, but separate as the balloon is inflated, analogous to how the device would operate if a bare metal or polymer plurality of stents was instead employed.

The coverings as described herein may be delivered with the devices. They provide an interconnection between the rings of the plurality of devices without imparting the stiffness of axial connector portions such as tie bars. The coverings, when present, are delivered with the ring-shaped intraluminal support devices, and are disposed between the vessel wall and the outer surface of the devices.

A covering 60/70/160 as disclosed above may be made of a biocompatible material that is well-tolerated by the tissues it contacts. In some cases, it may be desirable for the material of the covering 60/70/160 to biodegrade during treatment, as after deployment and implantation it is no longer required in some instances to maintain spacing by an extrinsic means. Examples of such bioresorbable or biodegradable coverings include, but are not limited to, polylactic acid or polycaprolactone and their derivatives. These coverings may optionally be reinforced by other bioplastic fibers. In other embodiments, the covering may not be biodegradable. A non-biodegradable covering may be made of materials including, but not limited to, polytetrafluoroethylene (PTFE) and its derivatives, including esPTFE.

As a person skilled in the art will readily appreciate, the above description is meant as an illustration of implementation of the principles this application. This description is not intended to limit the scope of this application in that the system is susceptible to modification, variation and change, without departing from the spirit of this application, as defined in the following claims.

What is claimed is:

1. A medical device assembly comprising:
a balloon catheter comprising a catheter body extending from a proximal end to a distal end and defining a longitudinal axis therethrough, and a balloon inflatable from a deflated state to an inflated state disposed circumferentially around the catheter body; and
a plurality of separate intraluminal support devices for implantation into a lumen of a body vessel, each intraluminal support device comprising a tubular body comprising at least one ring and being radially expandable from a compressed state to an expanded state, each intraluminal support device being a self-expanding device;
the plurality of intraluminal support devices being releasably secured about a delivery portion of the balloon in the deflated state, the plurality of intraluminal support devices being arranged such that a portion of one of the intraluminal support devices is in contact with a portion of an adjacent intraluminal support device, the plurality of intraluminal support devices being detached from the balloon when in the inflated state.

2. The medical device assembly of claim 1, wherein each ring comprises a plurality of struts and bends arranged in a repeating pattern to define a plurality of peaks.

3. The medical device assembly of claim 2, wherein each of the plurality of peaks defines a flat surface in a plane perpendicular to the longitudinal axis.

4. The medical device assembly of claim 2, wherein the plurality of peaks of one intraluminal support device are arranged in phase with the plurality of peaks of an adjacent intraluminal support device.

5. The medical device assembly of claim 2, wherein the plurality of peaks of one intraluminal support device are arranged out of phase with the plurality of peaks of an adjacent intraluminal support device.

6. The medical device assembly of claim 1, wherein each intraluminal support device comprises a shape-memory material.

7. The medical device assembly of claim 1, further comprising a covering attached to and disposed over each of the plurality of intraluminal support devices.

8. The medical device assembly of claim 7, wherein the covering contacts a portion of an outer surface of each intraluminal support device.

9. The medical device assembly of claim 7, wherein the covering surrounds an outer surface of each intraluminal support device entirely.

10. The medical device assembly of claim 1, wherein each intraluminal support consists of a single ring.

11. The medical device assembly of claim 1, wherein each intraluminal support comprises a plurality of connected rings.

12. The medical device assembly of claim 1, wherein the delivery portion of the balloon is substantially cylindrical in the inflated state.

13. The medical device assembly of claim 1, wherein the intraluminal support devices are configured to become spaced apart when deployed to a body vessel.

14. A medical device assembly comprising:
a balloon catheter comprising a catheter body extending from a proximal end to a distal end and defining a longitudinal axis therethrough, and a balloon inflatable from a deflated state to an inflated state disposed circumferentially around the catheter body; and
a plurality of separate self-expanding intraluminal support devices for implantation into a lumen of a body vessel, each intraluminal support device comprising a tubular body comprising at least one ring and being radially expandable from a compressed state to an expanded state, each ring comprising a plurality of struts and bends arranged in a repeating pattern to define a plurality of peaks, each of the plurality of peaks defining a flat surface in a plane perpendicular to the longitudinal axis; and
a covering attached to and disposed over each of the plurality of intraluminal support devices;
the plurality of intraluminal support devices being releasably secured about a delivery portion of the balloon in the deflated state such that at least one of the plurality of peaks of one of the intraluminal support devices is in contact with at least one of the plurality of peaks of an adjacent intraluminal support device, the plurality of intraluminal support devices being detached from the balloon when in the inflated state;
the delivery portion of the balloon being substantially cylindrical in the inflated state.

15. The medical device assembly of claim 14, wherein the plurality of peaks of one intraluminal support device are arranged in phase with the plurality of peaks of an adjacent intraluminal support device.

16. The medical device assembly of claim 14, wherein the plurality of peaks of one intraluminal support device are arranged out of phase with the plurality of peaks of an adjacent intraluminal support device.

17. The medical device assembly of claim 14, wherein the covering contacts a portion of an outer surface of each intraluminal support device.

18. The medical device assembly of claim 14, wherein the covering surrounds an outer surface of each intraluminal support device entirely.

19. The medical device assembly of claim 14, wherein each intraluminal support consists of a single ring.

20. The medical device assembly of claim 14, wherein each intraluminal support comprises a plurality of rings.

21. A method of making a medical device assembly, the method comprising:
aligning a plurality of self-expanding intraluminal support devices each comprising at least one ring comprising a plurality of struts and bends arranged in a repeating pattern to define a plurality of peaks and defining a lumen therethrough over a mandrel, the mandrel comprising a substantially cylindrical body having an outer surface, a plurality of pins extending outward from the outer surface, such that the plurality of pins constrain the plurality of intraluminal support devices in a predetermined arrangement comprising peak-to-peak contact between the intraluminal support devices;
removing the mandrel such that the plurality of intraluminal support devices remain in the predetermined arrangement;
inserting a balloon catheter through each lumen, the balloon catheter comprising a catheter body extending from a proximal end to a distal end and defining a longitudinal axis therethrough, and a balloon inflatable from a deflated state to an inflated state disposed circumferentially around the catheter body, the balloon comprising a delivery portion for retention and delivery of the plurality of intraluminal support devices; and
compressing the plurality of intraluminal support devices over the balloon in the deflated state.

22. The method of claim 21, wherein the predetermined arrangement comprises a peak of one intraluminal support device contacting a peak of an adjacent intraluminal support device out of phase.

23. The method of claim 21, comprising adhering the plurality of intraluminal support devices over the balloon in the deflated state by an adhesive.
